(19) United States
(12) Patent Application Publication
(10) Pub. No.: US 2004/0006393 A1
(43) Pub. Date: Jan. 8, 2004

(54) Implantable prosthetic knee for lateral compartment

(76) Inventor: Brian Burkinshaw, Pflugerville, TX (US)

Correspondence Address: Kenneth S. Barrow, Centerpulse USA Inc., Suite 1000, 12 Greenway Plaza, HOUSTON, TX, 77046, US

(21) Appl. No.: 10/347,942

(22) Filed: Jan. 21, 2003

Related U.S. Application Data
(60) Provisional application No. 60/393,862, filed on Jul. 3, 2002.

(51) Int. Cl. A61F 2/38

(52) U.S. Cl. 623/20.3; 623/20.32

(57) ABSTRACT

A unicompartmental meniscal prosthesis having a kidney-bean shape and being suitable for surgical implantation into the lateral compartment of the knee that is defined by the space between a femoral condyle and the respective tibial plateau. The prosthesis has fixation members at the posterior and anterior ends.

### CROSS REFERENCE TO RELATED APPLICATIONS

This application claims benefit of priority of U.S. Provisional Application Serial No. 60/393,862 filed Jul. 3, 2002.

### FIELD OF THE INVENTION

The disclosure herein relates generally to implantable orthopedic prostheses and, more particularly, to a unicompartmental implantable prosthetic knee for surgical implantation in the lateral compartment of the knee.

### BACKGROUND OF THE INVENTION

Various degenerative joint diseases can affect all compartments of the knee, including the medial and lateral compartments. Arthrosis, though, more commonly occurs in the medial compartment than in the lateral compartment. One reason for this occurrence is that the medial compartment experiences greater loads than the lateral compartment. For example, during normal gait, an adduction moment predominantly places forces in the medial compartment of the knee. Not surprisingly then, much attention has been devoted to designing a unicompartmental knee prosthesis for the medial compartment.

U.S. Pat. No. 6,206,927 entitled “Surgically Implantable Knee Prosthesis” teaches a unicompartmental knee designed to be implanted in the medial compartment between the femoral condyle and tibial plateau. This prosthesis comprises a kidney shaped body that is self-centering and devoid of any physical attachment means in the medial compartment. In use, the prosthesis rests in the medial dish of the femur and freely translates over the tibial plateau. The anatomical shape of the medial compartment and soft tissue balancing maintain the prosthesis in position and prevent it from being ejected from the medial compartment.

Unicompartmental prostheses currently used in the medial compartment of the knee, however, will not properly work in the lateral compartment. These prostheses, if left to translate freely in the lateral compartment, would likely be ejected or extruded from the lateral compartment. As one reason, the lateral compartment of the knee joint experiences different kinematic forces than the medial compartment. In particular, the medial compartment of the knee functions more as a primary pivot point during rotation between the femur and tibia in a normal flexion cycle. Normal anatomy of the medial compartment provides this pivot point because the central portion of the medial condyle is actually concave or slightly dished. By contrast, the lateral compartment of the knee exhibits much greater anterior-posterior translation between the femur and tibia. This translation can typically be between 11-20 mm. Radiographic analysis of the lateral compartment of the knee during deep flexion will actually show that the femur rolls off of the posterior aspect of the tibia. This movement occurs from the normal anatomical shape of the lateral compartment of the tibia. Specifically, the central portion of the tibial plateau transitions from a slight concave surface (on the anterior side) to a convex profile (centrally) and finally to a convex curvature (on the posterior side). This shape creates a cartilage lip over which the femur transitions during its full range of deep flexion.

Historically, a limited number of options exist to address diseases or disorders that exclusively affect the lateral compartment of the knee. If a patient has a painful lateral gonarthrosis, for example, a total knee arthroplasty (TKA), a high tibial osteotomy (HTO), or a supracondylar osteotomy of the femur could be performed. Both options are traumatic for the patient, and neither option is ideal, especially for a younger or active patient. During a supracondylar osteotomy, the surgeon removes a wedge of bone from the medial side of the femur, just above the gastrocnemius medial head, to correct the angular deformity at the joint line. Both segments of bone are then reattached with bone staples. Unfortunately, complications associated with this procedure have been high.

It therefore would be advantageous to provide a unicompartmental implantable orthopedic knee prosthesis that is adapted to be implanted in the lateral compartment of the knee and is shaped for the specific kinematic forces of this compartment.

### SUMMARY OF THE INVENTION

The present invention is directed to an unicompartmental meniscal prosthesis suitable for surgical implantation into the lateral compartment of the knee that is defined by the space between a femoral condyle and the respective tibial plateau. One important advantage of the present invention is that the prosthesis takes into account the specific kinematic forces of the lateral compartment and is specifically designed to be implanted in this compartment of the knee.

The prosthesis generally has kidney-bean or distorted elliptical shape in a plan view with a slightly elongated body that extends from an anterior side to a posterior side. Medial and lateral sides define two other sides of the body. The prosthesis also includes two major surfaces: An articulating surface and a bearing or fixation surface oppositely disposed from the articulation surface. The articulating surface has a central dish shape and overall is shaped and sized to track the lateral femur during flexion. By contrast, the bearing surface is more planar and is shaped and sized to abut the proximal end of the tibia. Both the anterior and posterior sides are generally rounded with a dome-like or elongated circular shape. The lateral side has a smooth, continuous, gradual curve that extends from the anterior to posterior sides. By contrast, the medial side has an “S” shaped curve with an indentation of the “S” shape located approximately in the center of the medial side.

The prosthesis is held in place on the tibia with limited fixation using two different fixation members. One fixation member is located at the posterior side and includes two hooks that extend downwardly from the bearing surface and curve inwardly toward the anterior side. The hooks are shaped to grab or hook bone at the proximal end of the tibia. The other fixation member is oppositely disposed from the first fixation member and is located at the anterior side of the prosthesis. This fixation member has a body portion with a curved generally rectangular shape that extends downwardly from the bearing surface. Two holes or bores extend through the body and are shaped to receive bone screws or fixation spikes. The body may be angled inwardly toward the posterior side in order to follow the natural contour of the tibia. These fixation members permanently fix the prosthesis to the tibial and prohibit movement while implanted. The proximal articulating surface of the spacer is designed to correct the medial joint space, yet allow the femur to translate freely through its full range of motion. This design will not allow the spacer to act as a free-floating body.

One advantage of the present invention is that the prosthesis will not be extruded or ejected from the lateral compartment of the knee. The fixation members firmly hold the prosthesis in the lateral compartment as the knee undergoes a full cycle of flexion.

Another advantage of the invention is that the prosthesis can be used to correct defects or disorders that affect the lateral compartment of the knee. As such, the prosthesis may obviate the need to perform complicated, traumatic surgeries (such as a TKA, HTO, or supracondylar osteotomy) to the lateral compartment. Further, the prosthesis provides for various types of fixation to the bone in order to provide stability to the prosthesis without destroying or significantly altering the original cartilage or subchondral bone of the tibial plateau or femoral condyle. In addition, no osteotomies or segments of bone are removed. Since the prosthesis uses limited fixation in the anterior and/or posterior aspects of the tibia, the original superior articulating surface reference points of the tibial bone structures are preserved to a great extent. The preservation of bone is important, especially if a revision or TKA is performed at a later date. One skilled in the art, though, will appreciate that limited superficial shaping of the contacting surfaces of the tibia and surrounding structures may be necessary to facilitate proper seating of the prosthesis. In addition, routine removal of osteophytes may be necessary to seat the component and/or facilitate improved range of motion for the soft tissue structures surrounding the joint.

As yet another advantage, the prosthesis of the present invention can be implanted in the lateral compartment and offered in a variety of different sizes and thicknesses. The prosthesis and variety of thicknesses can be used to correct deficiencies in spacing of the lateral knee joint.

### BRIEF DESCRIPTION OF THE DRAWINGS

FIG. 1 is a top perspective view of the unicompartmental meniscal prosthesis of the present invention.

FIG. 2 is a bottom perspective view of the unicompartmental meniscal prosthesis of FIG. 1.

FIG. 3 is a top view of the unicompartmental meniscal prosthesis of FIG. 1.

FIG. 4 is a bottom view of the unicompartmental meniscal prosthesis of FIG. 1.

FIG. 5 is a perspective view from the posterior end of the unicompartmental meniscal prosthesis of FIG. 1.

FIG. 6 is a perspective view from the anterior end of the unicompartmental meniscal prosthesis of FIG. 1.

FIG. 7 is a side perspective view from the medial side of the unicompartmental meniscal prosthesis of FIG. 1.

FIG. 8 is a side perspective view of a partial knee joint with the meniscal prosthesis of FIG. 1 implanted in the lateral compartment.

FIG. 9 is an enlarged plan view of the tibia and fibula of FIG. 8 with the meniscal prosthesis of FIG. 1 implanted in the lateral compartment.

FIG. 10 is a bottom perspective view of an alternate embodiment of a meniscal prosthesis.

FIG. 11 is a side perspective view from the medial side of an alternate embodiment of a meniscal prosthesis.

FIG. 12 is an end perspective view from the anterior side of the meniscal prosthesis of FIG. 11.

FIG. 13 is a side perspective view from the medial side of another alternate embodiment of a meniscal prosthesis.

FIG. 14 is an end perspective view from the anterior side of the meniscal prosthesis of FIG. 13.

### DETAILED DESCRIPTION

The meniscal prosthesis of the present invention is a unicompartmental prosthesis adapted to be implanted into the knee using arthroscopic and minimally invasive surgical techniques known to those skilled in the art. The prosthesis is adapted to be positioned within a lateral compartment of the knee in which a portion of the natural meniscus is ordinarily located. The natural meniscus may be maintained in position or may be wholly or partially removed, depending upon its condition. Under ordinary circumstances, pieces of the natural meniscus that have been torn away are removed; and damaged areas of the meniscus may be trimmed as necessary. In other instances, the entire portion of the meniscus residing in the meniscal cavity may be removed. Thus, the term “meniscal prosthesis” is descriptive of the location of the prosthesis rather than implying that it is a replacement for, or has the shape of, the natural meniscus. Actually, as described hereinafter, the shape of the meniscal prosthesis is not the same as the natural meniscus.

Further, as noted, the meniscal prosthesis of the present invention is unicompartmental. The term “unicompartmental” means that the prosthesis is adapted for implantation into but one compartment defined by the space between a femoral condyle and its associated tibial plateau. In other words, the prosthesis is not a “bicompartmental” prosthesis that, in one rigid prosthesis, could be inserted into both of the two femoral condyle/tibial plateau compartments. Specifically, the prosthesis of the present invention is adapted to be inserted into the lateral compartment of the knee and not the medial compartment.

Contrary to other prostheses that are composed of soft, compliant material designed to assume the function of the natural meniscus, the prosthesis of the present invention is composed of relatively hard, relatively high modulus material. Suitable materials are, for example, steel, ceramics, and reinforced and non-reinforced thermoset or thermoplastic polymers. The prosthesis need not be made of a single material, but composite structures of steel/thermoplastic, steel/ceramic, ceramic/polymer, etc., may be used.

Generally when composite structures are used, portions of the prosthesis expected to have the most wear or highest stress may be made of stronger, more abrasion resistant material than the remaining portions. This method may be ideal for use in conjunction with cultured chondrocyte implantation (cartilage cells used as seeds) or osteochondral transplantation or mosaicplasty. Moreover, when the locus of damage to the articular cartilage or to portions of the bone structure are known, the relatively constant radius of the surface of the meniscal prosthesis will bridge the defective areas and, thus, redistributing load to healthy tissue. This redistribution may allow inflamed, diseased, or other damaged areas to regenerate.

Alternatively, the present invention may be used with biologically active substances. These substances may contain pharmaceutical agents to stimulate cartilage growth or retard cartilage degeneration. Further, the surface of the present invention evenly distributes loads over regions of healthy articular cartilage, in general, abutting and bridging surfaces where articular cartilage degeneration or damage has occurred. As such, active substances may be applied at once or in a timed-release manner to the degenerated or damaged articular cartilage surface by means of, or in conjunction with, the meniscal prosthesis. The regenerating tissue will have time to mature and cross-link into a fully developed matrix. Moreover, as regeneration proceeds, the regenerating tissue will assume a shape dictated by the shape of the meniscal load-distributing prosthesis.

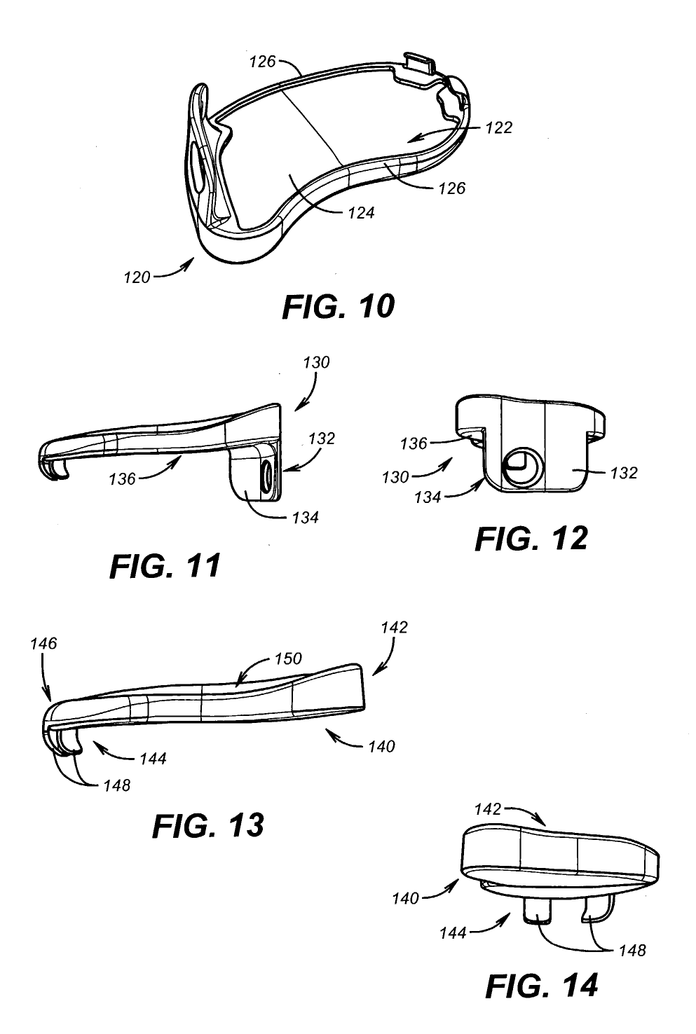

These biologically active substances may also be contained in a portion of the meniscal prosthesis itself (such as the prosthesis shown in FIG. 10). The portion may be filled with medication, or may be filled with a gel, paste, or soft polymer material that releases medication over a period of time. Preferably, this medically active portion actually contacts, or minimally contacts, the damaged tissue. Coatings may also be of a gel, paste, or polymer containing time-release medicaments.

The purpose of the prosthesis of the subject invention is to achieve a span-like effect to bridge the defective areas. However, in a composite variation, any single component (like a bioactive material component) may be softer than the supporting material. Rather than deforming to distribute a load relatively equally on the mating surfaces, the meniscal prosthesis of the present invention can function as rigid, substantially non-deforming prosthesis that does not necessarily spread the load uniformly, but rather may concentrate the load upon desired points, spanning areas of imperfection. If a soft and/or low modulus elastomer or thermoplastic is used for the entire prosthesis, the load is not concentrated on healthy tissue, and damaged areas due to wear and/or degeneration will be subjected to loading, decreasing the opportunity for the body's natural regenerative capability to function.

The high modulus of the meniscal prosthesis thus allows for the provision of recessed or non-contacting areas of the prosthesis to encourage articular cartilage regeneration. In softer, lower modulus materials, the naturally occurring loads will cause the softer prosthesis to deform and allow ordinarily non-contacting areas to contact bone or cartilage.

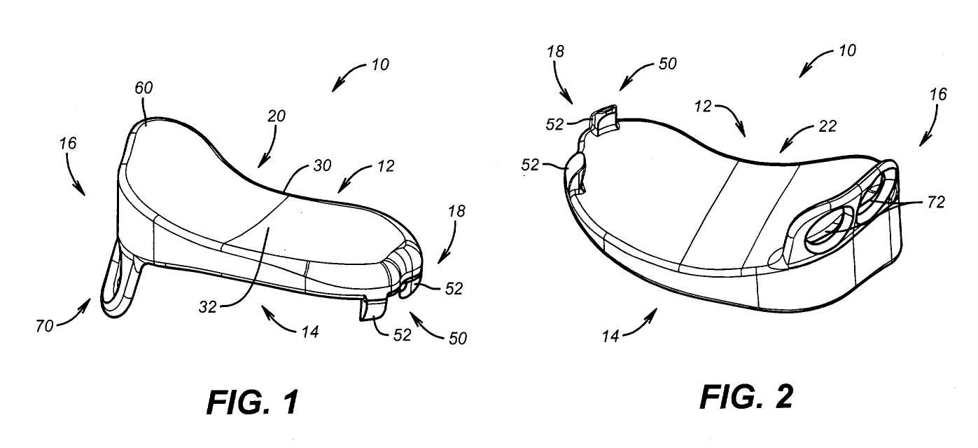

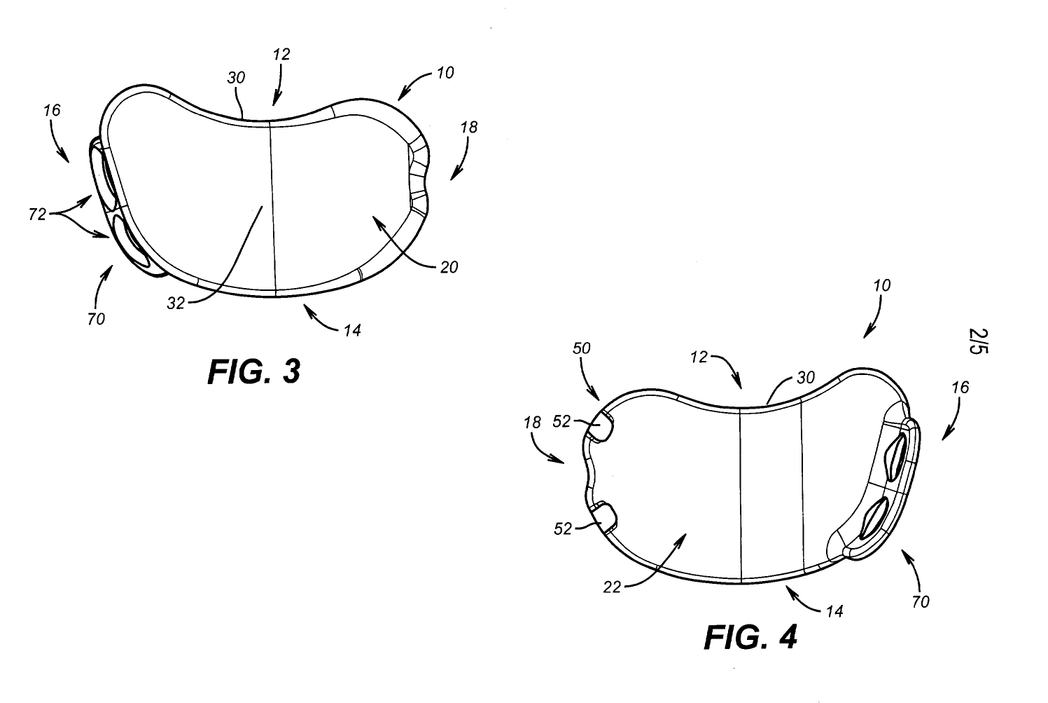

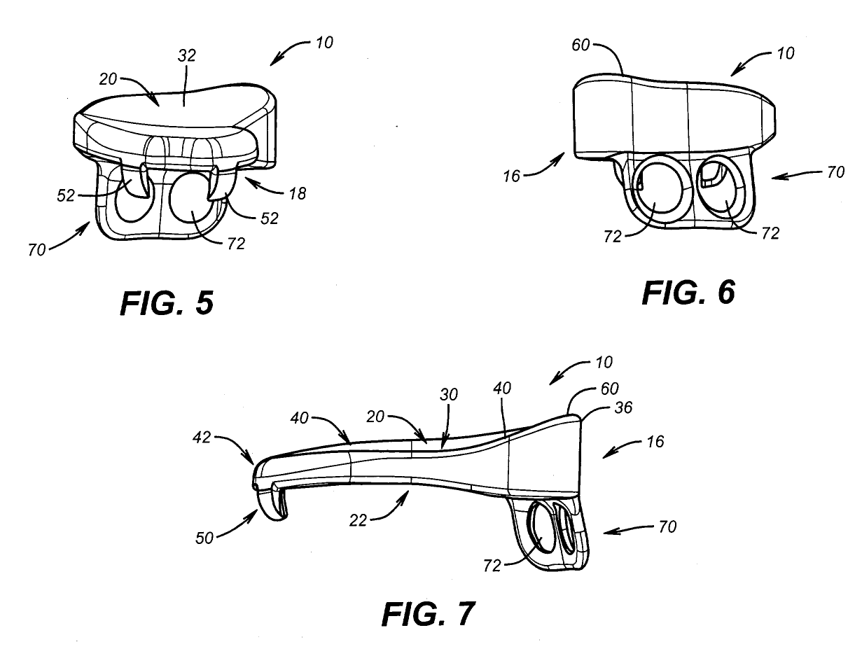

Turning now to FIGS. 1-7, the meniscal prosthesis 10 of the present invention is shown in detail. In a plan view, the prosthesis generally has a body with four sides or portions: A medial side 12, a lateral side 14, an anterior side 16, and a posterior side. Further, the body has two major surfaces: An articulating surface 20 and a fixation or bearing surface 22. Preferably, the body is formed as a single, unitary member with all members integrally formed with the body.

The prosthesis is generally shaped similarly to a kidney bean with a body that has an elongated shape from anterior 16 to posterior 18. Lateral side 14 has a convexly rounded shape, while medial side 12 is rounded with an indentation 30 along an outer edge. Further, articulation surface 20 of prosthesis 10 has a general dish shape in the proximal central, radial portion 32. The surface also has a polished, radial condylar track that is shaped to represent the radial sweep that the lateral femur is known to make during flexion. As best shown in FIG. 7, articulating surface 20 has a shape generally similarly to a “French Curve” drafting tool. The anterior edge is slightly rounded and protrudes vertically to a rounded edge in the first quarter region 36. This region then proceeds into a saddle shaped S-curve and creates the dish 30 near the center. The edge then slightly curves upwardly in the three-quarter region 40 and ends with a steep dropping curve 42 that projects down posteriorly, below the dish elevation. This downwardly sloping curve 42 forms a posterior fixation member 50.

The posterior fixation member is shaped as two spaced curved hooks 52. These hooks slightly curve under the bearing surface 22 toward the anterior side 16. Hooks 52 are shaped to match the radial profile of the posterior tibia. As such, the hooks are adapted to hook and engage the tibia at or below the region of the lateral capsular attachment on either side of the groove for the popliteus tendon.

The anterior side 16 includes the thickest portion of the prosthesis. This side has a lip 60 that extends upwardly toward the articulating surface 20. The lip includes a smooth receding surface that forms an edge for an anterior-superior cap for the tibia. Lip 60 can be shaped to wrap partially around the anterior lateral aspect of the proximal tibia.

The anterior side 16 also includes an anterior fixation member 70. The fixation member extends downwardly from the edge of the bearing surface 22 and has a generally curved rectangular shape or body. This curved shape follows or tracks the shape of the outer perimeter of the anterior side 16. Two fixation sites 72, shown as bores, extend through the body of the fixation member 70. Each bore is sized and shaped to receive a bone screw or fixation spike that enables the prosthesis 10 to be permanently fixed to tibial bone. Placement of one or more bone screws through the fixation member 70 and into bone prevents the prosthesis from lifting off the tibia when a posterior load is applied by the femoral condyle during deep knee flexion.

As shown in the figures, fixation member 70 can be angled with respect to the bearing surface 22. Specifically, the fixation member extends downwardly from the bearing surface and away from the posterior side 18. As such, an obtuse angle is formed between the fixation member 70 and the bearing surface 22. The degree of this angel can vary and should be sized to fit the natural contour of the proximal anterior surface of the tibia. The angle, for example, can range from about 91° to about 115°.

The length of the prosthesis 10 in an anterior-posterior direction is variable and can be made in a range of anatomic sizes. These lengths range from about 3 cm to 7 cm. Further, the width in a medial-lateral direction is variable and can be made in a range of anatomic sizes. These widths range from about 1 cm to 4 or 5 cm.

The thickness of the prosthesis can vary depending on various sizes. For example, the inside or central thickness may range from about 0.5 mm to 15 mm. Likewise, the edge or perimeter of the prosthesis can have a wide range of thickness, with the thickest portion occurring along the anterior side 16.

The actual shape and size of the meniscal prosthesis may be tailored to the individual. Individuals with high varus deformation due to wear, degeneration, or disease, may require a meniscal prosthesis that is of considerably greater thickness over the portions where wear is most advanced. In other patients, where trauma-induced damage rather than severe wear or degeneration has occurred, differences in the thickness, of the prosthesis will be more moderate. In general though, the prosthesis is elliptical or kidney-shaped when viewed from above, has rounded corners or edges, and has a thickness along the periphery that is greater than the thickness along the center of the prosthesis.

The bearing or fixation surface 22 is much more planar than the articulation surface 20 and is sized and shaped to rest on the proximal end of the tibia. The surface can have various configurations and textures that are adapted to meet individual needs of the patient. For instance, fixation surface 22 can have a smooth surface layer or can be adapted to directly engage and integrate with tibial bone with or without bone cement. Further, the surface can be, in whole or part, textured to promote osseointegration. If such integration with bone is desired, then various coatings known in the art (such as HA, ceramic, calcium phosphates, Cancellous Structured Titanium (CSTi), or porous metals) can be added to the fixation surface.

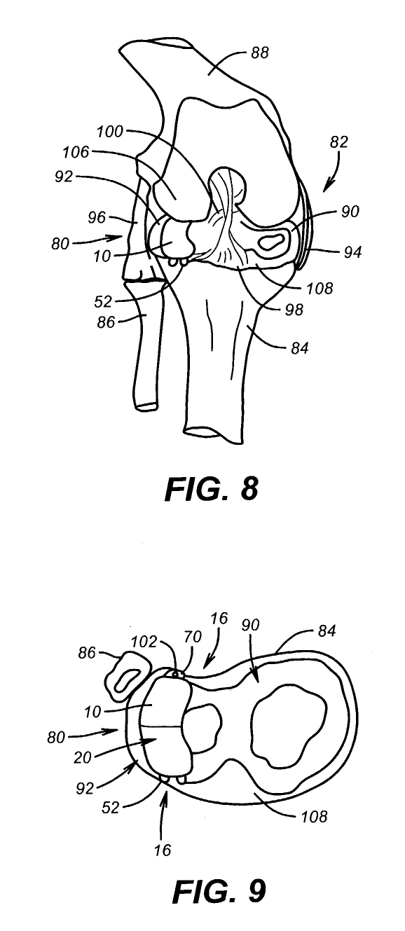

Turning now to FIGS. 8 and 9, the prosthesis 10 is shown implanted in lateral compartment 80 of a knee joint 82. These figures show three bones (a tibia 84, a fibula, 86, and a femur 88), two menisci (a medial meniscus 90 and lateral meniscus 92), and four ligaments (medial collateral ligament 94, lateral collateral ligament 96, anterior cruciate ligament 98, and posterior cruciate ligament 100). As shown, a bone screw or fixation spike 102 is connected to the anterior fixation member 70 to connect the anterior side 16 to the tibia. Hooks 52 hook into and engage the proximal posterior aspect of the tibia to secure the posterior side 18 of the prosthesis. The prosthesis is positioned between the femoral condyle 106 and tibial plateau 108 such that the articulating surface 20 is adapted to articulate with the femoral condyle and the bearing surface is adapted to rest adjacent the tibial plateau.

One advantage of the present invention is that the meniscal prosthesis, when implanted, can fix or aid an imbalance of the soft tissue and/or erosion of the joint space in the lateral compartment. Such an imbalance can cause a “knock-knee” stance or genu valgum. The meniscal prosthesis can restore the joint to have a more natural balance.

One skilled in the art will appreciate that the embodiments of the prosthesis can be altered without departing from the scope of the invention. FIGS. 10-14 illustrate examples of such altered embodiments.

FIG. 10 shows an alternate meniscal prosthesis 120 generally configured like the prosthesis 10 shown in FIGS. 1-7. Prosthesis 120, though, has a bearing surface 122 with a pocket or cavity 124. A rim or shoulder 126 extends around the bottom perimeter of the body of the prosthesis 120. This shoulder defines the boundary of the pocket 124. This pocket can be provided with a biological active substance, as discussed in connection with FIGS. 1-7, to induce bone integration, cartilage formation, or the like. Additionally, bone-void filling substances such as polymethyl methacrylate (PMMA) and calcium phosphate bone void fillers may be placed in this area to stimulate additional attachment to the tibial surface. PMMA is a common “bone cement”, well known by those skilled in the art. Calcium phosphate bone void fillers and cements are typically intended to provide a temporary, resorbable biologic scaffold used in the formation of new bone. Such an application of calcium phosphate bone void filler could be utilized with this implant in conjunction with other procedures to create a natural biologic bond between the tibia and the implant over time.

FIGS. 11 and 12 show another alternate meniscal prosthesis 130 generally configured like the prosthesis 10 shown in FIGS. 1-7. Prosthesis 130, though, has an anterior fixation member 132 that includes a single bore or hole adapted to receive a bone screw or fixation spike. Further, the body 134 of the fixation member is perpendicular to the bearing surface 136. As such, an obtuse or acute angle is not formed between the body of the fixation member and bearing surface.

FIGS. 13 and 14 show yet another alternate meniscal prosthesis 140 generally configured like the prosthesis 10 shown in FIGS. 1-7. Prosthesis 140, though, does not have a fixation member at the anterior side 142. A single fixation member 144 extends downwardly at the posterior side 146 of the prosthesis. This fixation member is identical to the fixation member 50 described in connection with FIGS. 1-7. Prosthesis 140 has relatively minimal fixation in the posterior aspect and, as such, can be constrained by the posterior capsule, and more importantly by the position of the popliteus tendon, located between the two posterior hooks 148. The articulating surface of the femur, in intimate contact with the conforming articulating surface 150 of the prosthesis will prevent lateral and or anterior subluxation of the prosthesis.

Although illustrative embodiments have been shown and described, a wide range of modifications, changes, and substitutions is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A meniscal prosthesis implantable in a knee joint, the meniscal prosthesis comprising:
a body adapted to be implanted in a lateral compartment of the knee joint between a femoral condyle and a tibial plateau and having an articulating surface adapted to articulate with the femoral condyle, a bearing surface adapted to rest adjacent the tibial plateau, and a periphery defined by lateral, medial, anterior, and posterior sides, the body further having a kidney-bean shape and a first fixation member being adapted to engage tibial bone to permanently fix the body to the bone.

2. The meniscal prosthesis of claim 1 wherein the first fixation member extends downwardly from the bearing surface at the posterior side.

3. The meniscal prosthesis of claim 2 wherein the first fixation member curves under the bearing surface toward the anterior side.

4. The meniscal prosthesis of claim 3 wherein the first fixation member includes two spaced apart hooks.

5. The meniscal prosthesis of claim 2 wherein the body includes a second fixation member different than the first fixation member.

6. The meniscal prosthesis of claim 5 wherein the second fixation member extends downwardly from the bearing surface at the anterior side.

7. The meniscal prosthesis of claim 6 wherein the first fixation member includes at least one hook, and the second fixation member includes a body portion adapted to receive a bone screw.

8. The meniscal prosthesis of claim 7 wherein the body portion of the second fixation member has a curved rectangular configuration with at least one bore extending through the body portion.

9. A implantable prosthesis, comprising:
an elliptically shaped body implantable in a lateral compartment of a knee joint between a femoral condyle and a tibial plateau of a tibia, the body having an articulating surface adapted to articulate with the femoral condyle, a bearing surface oppositely disposed from the articulating surface and adapted to rest adjacent the tibial plateau, and at least one fixation member adapted to permanently connect the body to the tibia.

10. The implantable prosthesis of claim 9 wherein the prosthesis in plan view has a kidney-bean shape.

11. The implantable prosthesis of claim 9 wherein the fixation member extends downwardly from the body along an anterior side.

12. The implantable prosthesis of claim 11 wherein the fixation member has at least one bore adapted to receive a bone screw.

13. The implantable prosthesis of claim 12 wherein the fixation member has two spaced apart bores adapted to receive bone screws.

14. The implantable prosthesis of claim 11 wherein the fixation member forms an obtuse angle with the bearing surface.

15. The implantable prosthesis of claim 14 wherein the obtuse angle ranges from about 91° to about 115°.

16. A meniscal prosthesis implantable in a knee joint, the meniscal prosthesis comprising:
a body adapted to be permanently fixed in a lateral compartment of the knee joint between a femoral condyle and a tibial plateau of a tibia and having an articulating surface adapted to articulate with the femoral condyle, a bearing surface adapted to rest adjacent the tibial plateau, and a periphery defined by lateral, medial, anterior, and posterior sides, the body further having a first fixation member being adapted to engage tibial bone.

17. The meniscal prosthesis of claim 16 wherein the first fixation member is at the posterior side, and wherein the body further includes a second fixation member at the anterior side.

18. The meniscal prosthesis of claim 17 wherein the first and second fixation members are different from each other.

19. The meniscal prosthesis of claim 16 wherein the bearing surface includes a pocket adapted to receive a biological active substance.

20. The meniscal prosthesis of claim 19 wherein the biological active substance induces bone and cartilage growth.
